(19) United States
(12) Patent Application Publication
(10) Pub. No.: US 2006/0217629 A1
(43) Pub. Date: Sep. 28, 2006

(54) Portable communication device

(75) Inventors: Chao-Mao Huang, Taipei (TW); Yu-Sheng Chen, Taipei (TW)

Correspondence Address: BRUCE H. TROXELL, SUITE 1404, 5205 LEESBURG PIKE, FALLS CHURCH, VA, 22041, US

(73) Assignee: Inventec Appliances Corp.

(21) Appl. No.: 11/088,817

(22) Filed: Mar. 25, 2005

(51) Int. Cl. A61B 5/05

(52) U.S. Cl. 600/547

(57) ABSTRACT

A portable communication device combining body fat analyzer is disclosed, which is used to measure a value of body fat of a user thereof. A power source provides a first detecting current. A first detection node and a second detection node are located on a surface of the portable communication device and connected to the power source and contacted with the user, respectively. A current detecting module measures a second detecting current flown at the second detection node. A computing module generates a value of body fat of the user by computing the first and second detecting currents. A communication module transmits the generated value of body fat to a database at a distant end for a specific purpose.

### BACKGROUND OF THE INVENTION

1. Field of Invention

The present invention relates to a portable communication device, and particularly to a portable communication device combining a body fat analyzer.

2. Related Art

Portable communication devices, such as mobile phones, smart-phones and personal digital assistants (PDAs) having capability of communication, have been being under rapid development and widely used and thus facilitated circulation of information in our daily life.

In the age of automation, people who work in the offices and exercise little are more and more. As everybody knows, a small quantity of exercise often incurs unhealthy body. Obesity is a most direct result when people seldom do exercise and generally considered a potential factor of getting sick. Therefore, body fat is required to be measured on a regular basis.

A trend of variation of body fat is generally concerned and thus required to be observed to see if any sudden increase or decrease is occurred. To clearly grasp a level of body fat, a body fat analyzer is generally relied on.

For a prior body fat analyzer, a testee has to be measured with a large instrument for medical use by a professional in this field. In performing this measurement, thickness of subcutaneous fat and conversed weight in water environment are required to be known. Further, the X-ray absorption method is needed to be used in the measurement, which is performed from top to bottom with respect to the testee.

However, the prior body fat analyzers generally have the shortcomings of heavy volume, poor portability and pricy cost. Further, these body fact analyzers may only measure an average of body fat distributed in whole of the body and may not measure body fat at a specific part of the body.

For the latest body fat analyzers, bioelectrical impedance analysis is relied on in performing the measurement of body fat. The bioelectrical impedance analysis is performed based on relationship between impedance and water-containing level. Human's body is generally classified into fat organization and non-fat organization. In the fat organization, there is almost no water contained and presents a greater impedance. On the other hand, the non-fat organization, such as muscle and blood, contains a great deal of water and thus exhibits a lesser impedance.

In performing the bioelectrical impedance analysis, a low current of high frequency is provided and flown through a testee's body and an impedance of the body is obtained. Thereafter, the obtained value of impedance is directed to a microprocessor for some processing. Then, the processed value of impedance is compared with data stored in a database built for a similar purpose and a ratio of the fat organization and the nonfat organization of the testee's body is computed and obtained. The Ministry of Health, Labour and Welfare of Japan has established a related database which is made with respect to the yellow race and the database or the like may be used as the above database.

Therefore, there is a need to provide a portable communication device combining a body fat analyzer so that a user thereof may perform a regular measurement over his body fat and avoid the above drawbacks generally existed in the prior body fat analyzers. SUMMARY OF THE INVENTION

It is, therefore, an object of the present invention to provide a portable communication device communication device combining so that a user thereof may measure his body fat at any time and any place. Also, the user of the portable communication device may measure body fat at some specific parts of the body. Further, information associated with the measured value of body fat may be transmitted to a distant end through a communication module of the portable communication device so that the information may be built in a database and monitored simultaneously and over a long time.

It is another object of the present invention to provide a monitoring module in in the portable communication device which may issue a warning message when a value of body fat of the user of the portable communication device is measured and presented as having a large variation so that the user may be reminded with his attention-worthy healthy condition.

To achieve the above objects, the portable communication device according to the present invention comprises a power source providing a first detecting current, a first detection node disposed on a surface of the portable communication device and to receive the first detecting current form the power source and to contact a user's first portion;, a second detection node disposed on the surface of the portable communication device and to contact the user's second portion, a current detecting module connected at one end connected to the second detection node and connected at the other end to the power source and detecting a second detecting current received via the second detection node when the user's second portion contacts the second detection node connecting to the power sppower spurcement atorst detection current and the other end a communication module, a storing m, a computing module computing body fat of the user according to the first and second detecting currents, a communication module transmitting information associated with body fat of the user generated by the computing module to a distant database, a storing module storing the information of body fat of the user and a monitoring module computing an average of a plurality of values of body fat of the user, comparing the computed average with the latest value of body fat computed by the computing module and issuing a warning message when a difference between the average and the latest value of body fat obtained from the computing module is greater than a specific value.

With the portable communication device, the objects of measuring body fat at any time and any place, measuring body fat at some specific parts of the body, transmitting the measured value of body fat to a distant end for being putting in an archive and monitoring simultaneously and over a long time.

The advantages and spirits of the present invention may be further understood after the following detailed description is read with reference to the accompanying drawings.

### BRIEF DESCRIPTION OF THE DRAWINGS

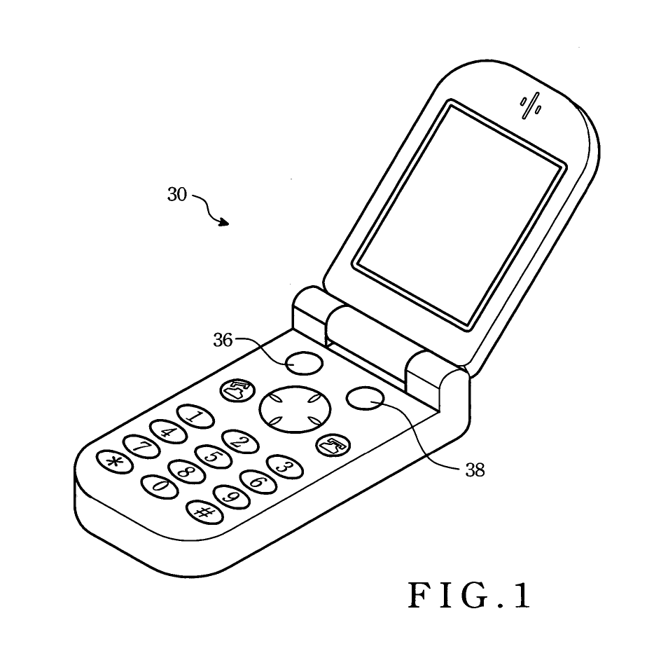

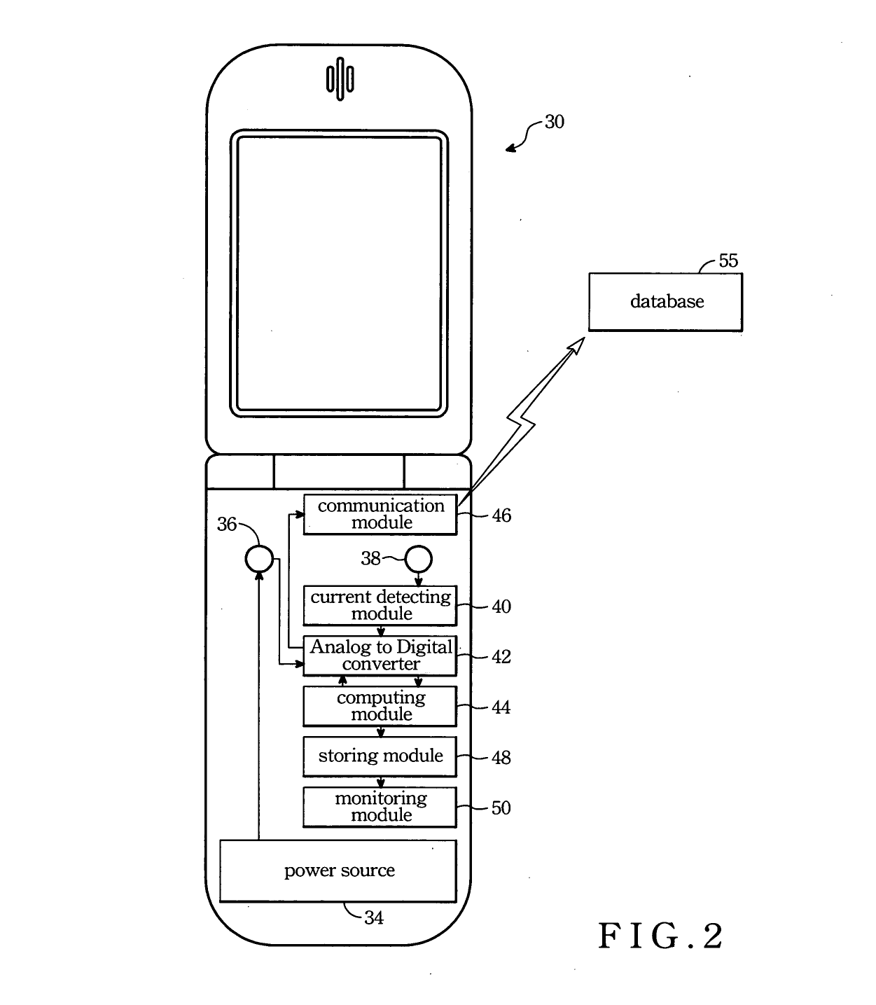

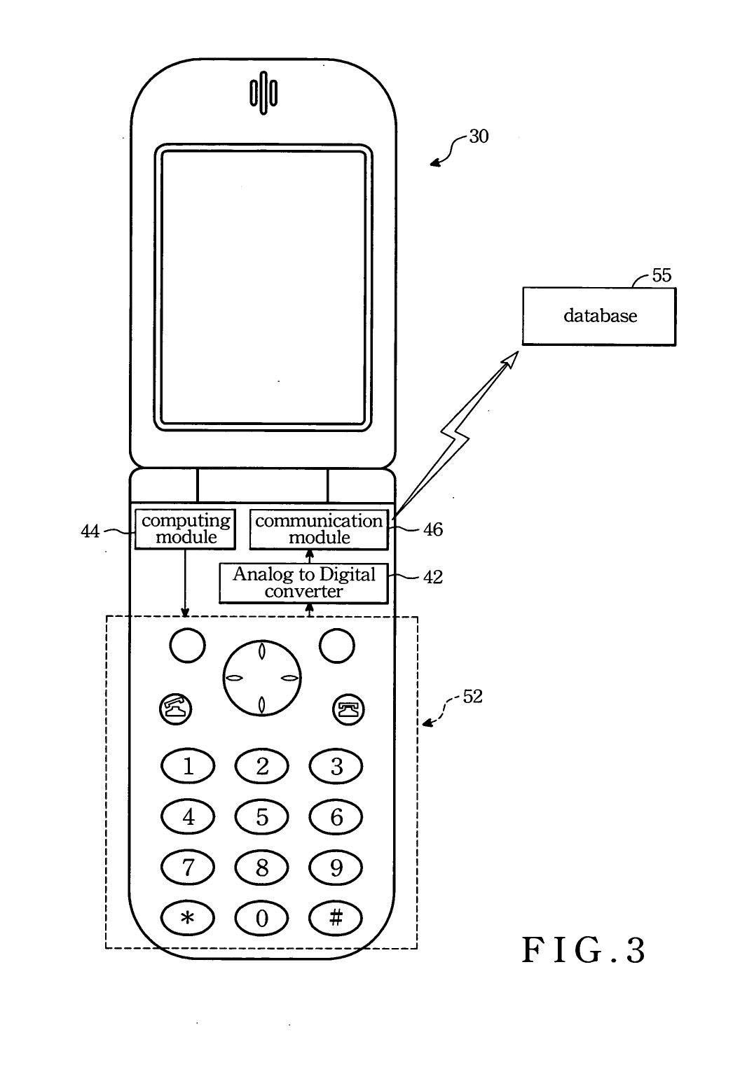

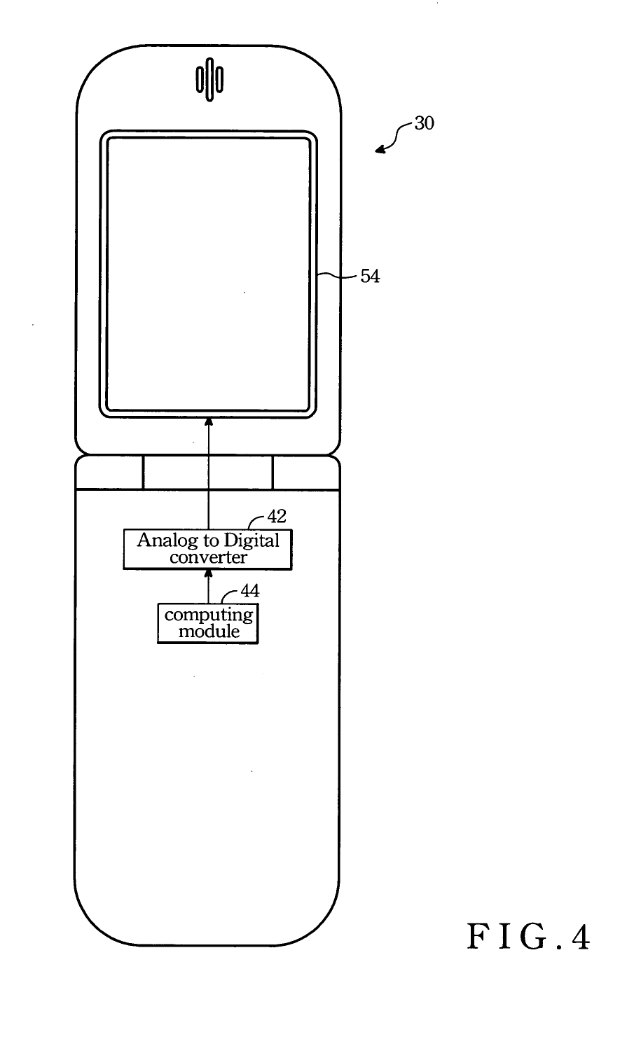

The invention will become more fully understood from the detailed description given hereinbelow illustration only, and thus are not limitative of the present invention, and wherein: FIG. 1 is a schematic diagram of an outline of a portable communication device according to the present invention; FIG. 2 is a schematic diagram of a portable communication device according to the present invention; FIG. 3 is a schematic diagram of a keystroke module according to the present invention; and FIG. 4 is a schematic diagram of a displayer according to the present invention.

### DETAILED DESCRIPTION OF THE INVENTION

FIG. 1 is a schematic diagram of a portable communication device 30 according to the present invention. FIG. 2 is a schematic diagram of a portable communication device 30 according to the present invention. Referring to FIG. 1 and FIG. 2, the present invention is related to the portable communication device 30. As shown in FIG. 1, a first detection node 36 and a second detection node 38 are located on a surface of the portable communication device 30.

Referring to FIG. 2, the portable communication device 30 comprises a power source 34, a first detection node 36, a second detection node 38, a current detecting module 40, an Analog to Digital converter (ADC) 42, a computing module 44, a communication module 46, a storing module 48 and a monitoring module 50.

In operation, the power source 34 provides a current required for portable communication device 30 and provides a first detecting current to a first detection node 36. The power source 34 may be a battery of the portable communication device 30. In addition, there also provides a second detection node 38. The first and second detection nodes 36,38 are both located on the surface of the portable communication device 30. The first detection node 36 is electrically connected to the power source 34 at one end to receive the first detecting current while exposed on the communication device 30 at the other end to contact a testee's first portion, i.e. a user of the portable communication device 30. The second detection node 38 contacts the testee's second portion. The first and second portions of the testee may be, for example, thumbs of the left hand and the right hand of the testee, respectively.

The current detecting module 40 is electrically connected at one end to the second detection node 38 and connected at the other end to the power source 34. When the testee contacts the first and second detection nodes 36,38 at the first and second portions, a current loop is formed between the portable communication device 30 and the first and second portions of the testee. At this time, the second detection node 38 receives a second detecting current and the second detecting current is detected by the current detecting module 40, which may be an ammeter or the like.

The ADC 42 converses the first detecting current received via the first detection node 36 and the second detecting current at the second detection node 38 detected by the current detecting module 40 into a set of digital signals. The computing module 44 generates a value of body fat of the testee by computing the first and second detecting currents.

The computation principle which the computing module is relied on is that an impedance may be obtained from a supplied voltage and a detected current and the value of body fat may be generated by comparing the impedance with data built in a similar purpose-oriented database, such as that established by the Ministry of Health, Labour and Welfare of Japan or a self-established database.

The communication module 46 is a radio frequency (RF) module used in the portable communication device 30 and needs not to be additionally built in body fat module 32. Before sent to the communication module 46, the generated value of body fat from the computing module 44 is first conversed to an analog signal by the ADC 42 so that the value of body fat may be transmited. Next, the communniaction module 46 transmits the analog signal of the value of body fat to a database 35 located at a distant end and the value of body fat is stored in the database 35 with the corresponding portable communication device 30 as an index, such as a telephone number of the portable communication device 30. The value of body fat of the testee stored in the database 35 may be monitored simultaneously over a long period of time for a specific purpose, such as used for a health report.

After the value of body fat is generated from the computing module 44, the computed value of body fat may be first stored in the storing module 48. Next, the monitoring module 50 is provided to compare an average of a plurality of measurements of body fat of the user with the latest value of body fat of the user obtained from the computing module 44. When the latest value of body fat is greater than a specific value, the monitoring module 50 may issue a warning message to inform the user that his body fat has become exceptional or the like. The warning message may be issued through, for example, a speaker of the portable communication device 30, flickering a frame of a displayer of the portable communication device 30 or displaying some specific texts or symbols.

Referring to FIG. 3, a schematic diagram of a keystroke module according to the present invention is illustrated therein. Since the portable communication device 30 may have a plurality of users, the keystroke module 52 may be used to input the users' information corresponding to their values of body fat. For example, when David Wang's value of body fat is displayed and he desires to store the value, then the name “David Wang” is required to be input through the keystroke module 52 and the value is stored under David Wang's catalogue.

Referring to FIG. 4, a schematic diagram of a displayer according to the present invention is illustrated therein. The displayer 54 equipped on the portable communication device 30 may be provided for displaying the latest value of body fat after conversion of ADC 44. Additionally, the plurality of values of body fat stored in the storing module 48 may be also displayed.

In the above, the storing module 48 may be a flash memory or the like. The operations of the computing module 44 and the monitoring module 50 may be edited as an application program (AP) respectively in Java, C, Assembly or any suitable program languages and pre-stored in a read only memory (ROM) or a flash memory. Coordinated by the microprocessor, the expected functions of the computing module 44 and the monitoring module 50 may be performed.

Therefore, by using the portable communication device 30, the user may perform a measurement of body fat at any time and any place. In addition, the user may measure his body fat at some specific parts of the body. Further, the user may transmit the measured value of body fat to a distant end for being put into an archive and monitoring simultaneously and over a long time. Furthermore, the user may be reminded with an exceptional value of body fat through the warning message issued by the monitoring module 50.

While the preferred embodiments of the present invention have been set forth for the purpose of disclosure, modifications of the disclosed embodiments of the present invention as well as other embodiments thereof may occur to those skilled in the art. Accordingly, the appended claims are intended to cover all embodiments which do not depart from the spirit and scope of the present invention.

1. A portable communication device capable of measuring body fat of a user thereof, comprising:
a power source providing a first detecting current;
a first detection node disposed on a surface of the portable communication device to receive the first detecting current form the power source and to contact a user's first portion;
a second detection node disposed on the surface of the portable communication device to contact the user's second portion;
a current detecting module connected to the second detection node and the power source and detecting a second detecting current received via the second detection node when the user's second portion contacts the second detection node; connecting to the power sppower spurcement atorst detection current and the other end a communication module, a storing m
a computing module computing a value of body fat of the user according to the first and second detecting currents; and
a communication module transmitting information associated with body fat of the user generated by the computing module to a database at a distant end.

2. The portable communication device as recited in claim 1, further comprising a storing module storing the information of body fat of the user.

3. The portable communication device as recited in claim 1, further comprising a monitoring module computing an average of a plurality of values of body fat of the user, comparing the computed average with the latest value of body fat computed by the computing module and issuing a warning message when a difference between the average and the latest value of body fat obtained from the computing module is greater than a specific value.

4. The portable communication device as recited in claim 1, further comprising an input module disposed on the portable communication device and used to be inputted with information associated with the user for being stored in the database.

5. The portable communication device as recited in claim 1, further comprising an analog to digital converter (ADC) conversing the first detecting current and the second detecting current to a set of digital signals to be computed by the computing module to generate the value of body fat.

6. The portable communication device as recited in claim 1, further comprising a displayer disposed on the portable communication device and displaying the value of body fat of the user.
